(19) United States
(12) Patent Application Publication
(10) Pub. No.: US 2007/0203441 A1
(43) Pub. Date: Aug. 30, 2007

(54) Semi-Custom Ankle Brace System

(76) Inventor: Ernesto G. Castro, Mesa, AZ (US)

Correspondence Address: STONEMAN LAW OFFICES, LTD, 3113 NORTH 3RD STREET, PHOENIX, AZ, 85012, US

(21) Appl. No.: 11/742,422

(22) Filed: Apr. 30, 2007

Related U.S. Application Data
(63) Continuation-in-part of application No. 10/641,887, filed on Aug. 14, 2003, now abandoned.
(60) Provisional application No. 60/403,945, filed on Aug. 15, 2002.

(51) Int. Cl. A61F 5/00

(52) U.S. Cl. 602/27; 602/5

(57) ABSTRACT

Disclosed are methods and apparatus of semi-custom ankle brace systems adapted to be heat-formed onto a patient's foot. The system laces around the lower calf, ankle, heel, and arch, and also provides compression and assists the unitary ankle support in preventing pronation, suppination, and plantarflexion. The boot is custom heat-molded to the end user by heating it to about one-hundred-eighty degrees and then applying it to the patient's foot while it cools. The system comprises a unitary, heat-moldable ankle and full arch support encased in soft inner and outer linings. The inner lining, outer lining, and unitary ankle support form a toeless boot with minimum bulk.

### CROSS-REFERENCE TO RELATED APPLICATIONS

The present application is a continuation-in-part of related application Ser. No. 10/641,887, filed Aug. 14, 2003, entitled “SEMI-CUSTOM ANKLE BRACE SYSTEM”, which is related to and claims priority from prior provisional application Ser. No. 60/403,945, filed Aug. 15, 2002, entitled “SEMI-CUSTOM ANKLE BRACE SYSTEM”, and is related to my issued U.S. utility Pat. No. 6,443,919, filed Jul. 27, 2000, entitled “CUSTOM ANKLE BRACE SYSTEM”, and is related to my issued U.S. utility Pat. No. 6,155,997, filed Dec. 16, 1997, entitled “CUSTOM ANKLE BRACE SYSTEM”, the contents all of which are incorporated herein by this reference and are not admitted to be prior art with respect to the present invention by the mention in this cross-reference section.

### BACKGROUND

Some people suffer from certain conditions of the feet that either greatly limit unaided mobility or require the use of aids such as walkers or canes in order for the individual to be mobile. Conditions which may cause this debilitating condition include tibialis tendinitis or rupture; degenerate joint disease; talocalcaneal varus or valgus; and severe pronation and/or trauma to ankle, subtalar area, or midtarsal. To treat these conditions necessarily requires a stabilizing-type apparatus in order to stabilize the ankle area, talocalcaneal, midtarsal, and subtalar joints, so that medial and lateral stability of the foot is achieved with the result that the patient enjoys the benefits of greater mobility.

Individual afflicted with these conditions may try to utilize mechanical devices that attempt to stabilize the foot so that the heel is more aligned with the bones in the lower leg. Generally, the braces used for these types of ailments are large, thick, cumbersome, and expensive. The patient must purchase a shoe or shoes that are one or many shoe sizes larger than they would normally require, or the patient must pay the expense of having a shoe made from a casting of their foot. Furthermore, less expensive, over-the-counter ankle braces do not provide adequate support, and chafe sensitive areas of the feet that do not conform to the off-the-shelf brace. Thus, a need exists to provide inexpensive, comfortable and easy to use braces.

### OBJECTS AND FEATURES OF THE INVENTION

A primary object and feature of the present invention is to fulfill the above-mentioned needs by the provision of a semi-custom ankle brace system overcoming the above-stated problems. A further primary object and feature of the present invention is to provide such a semi-custom ankle brace system that is efficient and permits the patient to wear essentially normal shoes. In addition, it is a primary object and feature of this invention to provide such a custom ankle brace system in connection with, and making use of, a novel semi-custom ankle brace.

Further, it is a primary object and feature of this invention to provide methods of fabricating novel ankle brace systems. Further, is a primary object and feature of this invention to provide methods of using novel ankle brace systems. Even further, it is a primary object and feature of this invention to provide novel business systems for use and retail distribution of ankle brace systems. Other objects and features of this invention will become apparent with reference to the following invention descriptions.

### SUMMARY OF THE INVENTION

In accordance with a preferred embodiment hereof, this invention provides a method of fabricating a semi-custom ankle brace in a preset number of sizes, comprising the steps of cutting at least one inner lining from at least one soft flexible material using at least one standard pattern of at least one preset size; attaching such at least one inner lining so that it contains at least one seam essentially parallel to the back of at least one heel from the bottom of the at least one heel to the top of such at least one semi-custom ankle brace; cutting at least one outer lining from at least one soft flexible material using at least one standard pattern of at least one preset size; attaching such at least one outer lining so that it contains at least one seam essentially parallel to the back of at least one heel from the bottom of the at least one heel to the top of such at least one semi-custom ankle brace; cutting at least one unitary stiff support, using at least one standard pattern of at least one preset size, from at least one sheet of at least one thermally formable material; forming such at least one unitary stiff support to the shape of at least one standard sized last; holding such at least one unitary stiff support between such at least one inner lining and such at least one outer lining of such at least one semi-custom ankle brace; attaching at least one perimeter of such at least one inner lining and such at least one outer lining together, so as to encase such at least one unitary stiff support; attaching at least one edge of at least one tongue to at least one lining of such at least one ankle brace system; and attaching at least one adjustably tightenable closure in such at least one ankle brace system.

Also, it provides such a method, wherein the step of attaching comprises the step of at least one of the group consisting essentially of stitching and gluing. In addition, it provides such a method, wherein the step of attaching at least one closure comprises the step of attaching at least one of the group consisting essentially of hook and loop fastener, elastic, and cinched strap. And, it provides such a method, wherein the step of attaching at least one closure comprises the step of inserting a plurality of eyelet grommets in such at least one ankle brace system and threading at least one lace through such plurality of eyelet grommets. Further, it provides such a method, further comprising the step of attaching such at least one inner lining and such at least one outer lining together around at least one portion of at least one perimeter of such at least one unitary stiff support. Even further, it provides such a method, wherein the step of attaching comprises the step of at least one of the group consisting essentially of stitching and gluing.

In accordance with another preferred embodiment hereof, this invention provides a method of using a semi-custom ankle brace system, comprising the steps of selecting at least one ankle brace system of at least one appropriate size to fit at least one foot of at least one patient; placing at least one thermally protective sock on at least one foot of at least one patient; submerging such at least one ankle brace system in at least one fluid at at least one temperature and for at least one time sufficient to soften the at least one thermoplastic material of such at least one ankle brace system; placing such at least one ankle brace system over such at least one thermally protective sock; tightening such at least one ankle brace system to the at least one foot of the at least one patient; moving the at least one foot and its bone structure to at least one preferred position and holding the at least one foot until such at least one thermoplastic material of such at least one ankle brace system cools; wherein such at least one ankle brace system is given at least one semi-custom fit to the at least one patient's at least one foot. Moreover, it provides such a method, further comprising the step of placing at least one compression sock over such at least one ankle brace system to apply compression evenly over the at least one foot. Additionally, it provides such a method, further comprising the step of placing at least one plastic bag over such at least one ankle brace system, prior to placing such at least one compression sock over such at least one ankle brace system.

In accordance with another preferred embodiment hereof, this invention provides a method used to retail ankle brace systems worn inside shoes, comprising the steps of providing to orthotics retailers at least one inventory listing of such at least one ankle brace systems in at least one range of sizes no greater than available shoe sizes; providing to such retailers at least one price at which such retailers may order and purchase such initial selection of such ankle brace systems; taking at least one first order from such retailers and providing to such retailers such at least one initial selection of such at least one ankle brace systems; and providing at least one training to such retailers in proper application and fitting, using conformation of low-temperature thermoplastic unitary stiff supports of such at least one ankle brace systems. Also, it provides such a method, further comprising providing to such retailers at least one price at which such retailers may re-order and purchase at least one additional stock of such at least one ankle brace systems. In addition, it provides such a method, further comprising taking at least one additional order from such retailers and providing to such retailers such at least one additional stock of such at least one ankle brace systems as required to replenish such retailers stocks. And, it provides such a method, wherein the step of providing at least one training to such retailers comprises the step of providing at least one in-person training. Further, it provides such a method, wherein the step of providing at least one training to such retailers comprises the step of providing at least one training material. Even further, it provides such a method, wherein the step of providing at least one training material further comprises the step of providing at least one video training material in the form of at least one of the group consisting essentially of DVD, CD-Rom, and videotape.

In accordance with another preferred embodiment hereof, this invention provides a system for stabilizing an ankle of a leg, having a lower calf having a medial surface and a lateral surface, and having a foot having an arch and a heel and a toes portion, comprising: unitary stiff support means for stiffly supporting the ankle in relation to the foot; wherein such unitary stiff support means comprises stiff portions comprising at least one medial portion to support the medial surface of the lower calf, at least one lateral portion to support the lateral surface of the lower calf, and at least one arch portion to support at least one substantial portion of the arch; wherein such unitary stiff support means further comprises essentially no stiff portions to support front surfaces of the lower calf, rear surfaces of the lower calf, top surfaces of the foot, nor toes portions of the foot. Moreover, it provides such a system, wherein such unitary stiff support means further comprises essentially no stiff portions structured and arranged to support the heel. Additionally, it provides such a system, further comprising inner liner means for lining between essentially all of such unitary stiff support means and such leg. Also, it provides such a system, further comprising outer liner means for lining outside and covering essentially all of such unitary stiff support means. In addition, it provides such a system, further comprising attachment means for attaching such inner liner means with such outer liner means in such manner as to hold essentially all of such unitary stiff support means within such inner liner means and such outer liner means. And, it provides such a system, further comprising adjustably tightenable closure means for adjustably closing such semi-custom ankle brace system around the ankle. Further, it provides such a system, further comprising gap closer means for closing at least one gap between such at least one medial portion and such at least one lateral portion.

In accordance with another preferred embodiment hereof, this invention provides a system used to stabilize an ankle of a leg, having a lower calf having a medial surface and a lateral surface, and having a foot having an arch and a heel and a toes portion, comprising: at least one unitary stiff support structured and arranged to stiffly support the ankle in relation to the foot, wherein such at least one unitary stiff support comprises stiff portions comprising at least one medial stiff portion structured and arranged to support the medial surface of the lower calf, at least one lateral stiff portion structured and arranged to support the lateral surface of the lower calf, and at least one arch stiff portion structured and arranged to support at least one substantial portion of the arch; wherein such at least one unitary stiff support further comprises essentially no stiff portions structured and arranged to support the front surface of the lower calf, the rear surface of the lower calf, the top surface of the foot, nor the toes portion of the foot. Even further, it provides such a system, wherein such at least one unitary stiff support further comprises essentially no stiff portions structured and arranged to support the heel. Moreover, it provides such a system, wherein such at least one unitary stiff support consists essentially of at least one plastic material. Additionally, it provides such a system, wherein such at least one unitary stiff support consists essentially of at least one thermoplastic material. Also, it provides such a system, wherein such at least one unitary stiff support consists essentially of at least one low-temperature-thermoplastic material. In addition, it provides such a system, wherein such at least one unitary stiff support consists essentially of at least one low-temperature-thermoplastic material comprising substantial malleability at about 180 degrees Fahrenheit. And, it provides such a system, wherein such at least one unitary stiff support comprises isoprene polymers. Further, it provides such a system, wherein such at least one unitary stiff support is approximately one eighth of an inch thick, before pre-forming. Even further, it provides such a system, further comprising at least one inner lining structured and arranged to lie at least between essentially all of such unitary stiff support and the leg. Moreover, it provides such a system, wherein such at least one inner lining comprises at least one flexible material. Additionally, it provides such a system, wherein such at least one flexible material comprises at least one of the group consisting essentially of fabric, padded nylon fabric, and leather. Also, it provides such a system, further comprising at least one outer lining, structured and arranged to lie at least outside of and covering essentially all of such at least one unitary stiff support. In addition, it provides such a system, wherein such at least one outer lining comprises at least one flexible material. And, it provides such a system, wherein such at least one flexible material comprises at least one of the group consisting essentially of fabric, padded nylon fabric, and leather.

Further, it provides such a system, further comprising: at least one inner lining structured and arranged to lie at least between essentially all of such at least one unitary stiff support and the leg; at least one outer lining, structured and arranged to lie at least outside of and covering essentially all of such at least one unitary stiff support; at least one attacher structured and arranged to attach such at least one inner lining with such at least one outer lining in such manner as to hold essentially all of such at least one unitary stiff support within such at least one inner lining and such at least one outer lining. Even further, it provides such a system, wherein such at least one attacher comprises at least one of the group consisting essentially of stitches and glue. Moreover, it provides such a system, further comprising at least one closure structured and arranged to adjustably tighten such semi-custom ankle brace system around the ankle. Additionally, it provides such a system, wherein such at least one closure comprises at least one of the group consisting essentially of grommet and lace, hook and loop fastener, elastic closure, and cinched strap. Also, it provides such a system, further comprising at least one tongue, structured and arranged to cover at least one gap under such at least one closure of such at least one semi-custom ankle brace system.

In addition, it provides such a system, wherein such at least one tongue comprises at least one flexible material. And, it provides such a system, wherein such at least one flexible material comprises at least one of at least one of the group consisting essentially of fabric, padded nylon fabric, and leather. Further, it provides such a system: wherein such at least one medial stiff portion is structured and arranged to be placed essentially adjacent a medial surface of the lower calf; wherein such at least one lateral stiff portion is structured and arranged to be placed essentially adjacent a lateral surface of the lower calf; wherein such at least one arch stiff portion is structured and arranged to be placed essentially adjacent at least one substantial portion of the arch; and wherein such at least one unitary stiff support further comprises essentially no stiff portions structured and arranged to be placed essentially adjacent a front surface of the lower calf, a rear surface of the lower calf, a top surface of the foot, nor the toes portion of the foot.

In accordance with another preferred embodiment hereof, this invention provides a system, for stabilizing an ankle of a leg, having a lower calf and a foot having an arch and a heel and a toes portion, comprising: at least one adjustably tightenable portion structured and arranged to essentially surround the lower calf; and at least one other adjustably tightenable portion structured and arranged to essentially tighten around the ankle, the heel, and the arch; wherein such at least one semi-custom ankle brace system is structured and arranged to be worn inside of at least one standard shoe large enough to hold such at least one semi-custom ankle brace system; wherein such at least one semi-custom ankle brace system comprises at least one unitary stiff support structured and arranged to stabilize the ankle in relation to the foot in such at least one semi-custom ankle brace system; and wherein such at least one unitary stiff support consists essentially of at least one low-temperature-thermoplastic material.

Even further, it provides such a system, wherein such at least one unitary stiff support consists essentially of at least one low-temperature-thermoplastic material comprising substantial malleability at about 180 degrees Fahrenheit. Even further, it provides such a system, wherein such unitary stiff support comprises stiff portions comprising at least one medial stiff portion structured and arranged to be placed essentially adjacent a medial surface of the lower calf, at least one lateral stiff portion structured and arranged to be placed essentially adjacent a lateral surface of the lower calf, and at least one arch stiff portion structured and arranged to be placed essentially adjacent at least one substantial portion of the arch; wherein such at least one unitary stiff support further comprises essentially no stiff portions structured and arranged to be placed essentially adjacent a front surface of the lower calf, a rear surface of the lower calf, a top surface of the foot, nor the toes portion of the foot. Even further, it provides such a system, wherein such at least one unitary stiff support further comprises essentially no heel stiff portion structured and arranged to be placed essentially adjacent to the heel.

In accordance with another preferred embodiment hereof, this invention provides a semi-custom ankle brace system structured and arranged to stabilize an ankle of a leg, having a lower calf having an inside (medial) surface and an outside (lateral) surface, and having a foot having an arch and a heel and a toes portion, comprising, in combination: at least one unitary stiff support structured and arranged to stiffly support the ankle in relation to the foot, wherein such at least one unitary stiff support comprises stiff portions comprising, at least one medial stiff portion structured and arranged to support the inside (medial) surface of the lower calf, at least one lateral stiff portion structured and arranged to support the outside (lateral) surface of the lower calf, and at least one arch stiff portion structured and arranged to support at least one substantial portion of the arch; wherein such at least one unitary stiff support further comprises essentially no stiff portions structured and arranged to support the front surface of the lower calf, the rear surface of the lower calf, the top surface of the foot, nor the toes portion of the foot.

Moreover, it provides such a semi-custom ankle brace system, wherein such at least one unitary stiff support further comprises essentially no stiff portions structured and arranged to support the heel. Additionally, it provides such a semi-custom ankle brace system, wherein such at least one unitary stiff support consists essentially of at least one plastic material. Also, it provides such a semi-custom ankle brace system, wherein such at least one unitary stiff support consists essentially of at least one thermoplastic material. In addition, it provides such a semi-custom ankle brace system, wherein such at least one unitary stiff support consists essentially of at least one low-temperature-thermoplastic material. And, it provides such a semi-custom ankle brace system, wherein such at least one unitary stiff support consists essentially of at least one low-temperature-thermoplastic material comprising substantial malleability at about 180 degrees Fahrenheit. Further, it provides such a semi-custom ankle brace system, wherein such at least one unitary stiff support consists essentially of ORTHOPLAST® thermoplastic. Even further, it provides such a semi-custom ankle brace system, wherein such at least one unitary stiff support is approximately one eighth of an inch thick, before pre-forming.

Moreover, it provides such a semi-custom ankle brace system, further comprising at least one inner lining structured and arranged to lie at least between essentially all of such unitary stiff support and the leg. Additionally, it provides such a semi-custom ankle brace system, wherein such at least one inner lining comprises at least one flexible material. Also, it provides such a semi-custom ankle brace system, wherein such at least one flexible material comprises at least one of the group consisting essentially of fabric, padded nylon fabric, and leather. In addition, it provides such a semi-custom ankle brace system, further comprising at least one outer lining, structured and arranged to lie at least outside of and covering essentially all of such at least one unitary stiff support. And, it provides such a semi-custom ankle brace system, wherein such at least one outer lining comprises at least one flexible material. Further, it provides such a semi-custom ankle brace system, wherein such at least one flexible material comprises at least one of the group consisting essentially of fabric, padded nylon fabric, and leather.

Even further, it provides such a semi-custom ankle brace system, further comprising at least one attacher structured and arranged to attach such at least one inner lining with such at least one outer lining in such manner as to hold essentially all of such at least one unitary stiff support within such at least one inner lining and such at least one outer lining. Moreover, it provides such a semi-custom ankle brace system, wherein such at least one attacher comprises at least one of the group consisting essentially of stitches and glue.

Additionally, it provides such a semi-custom ankle brace system further comprising at least one closure structured and arranged to adjustably tighten such ankle brace system around the ankle. Also, it provides such a semi-custom ankle brace system wherein such at least one closure comprises at least one of the group consisting essentially of grommet and lace, hook and loop fastener, elastic closure, and cinched strap.

In addition, it provides such a semi-custom ankle brace system, further comprising at least one tongue, structured and arranged to cover at least one gap under such at least one closure of such at least one semi-custom ankle brace system. And, it provides such a semi-custom ankle brace system, wherein such at least one tongue comprises at least one flexible material. Further, it provides such a semi-custom ankle brace system, wherein such at least one flexible material comprises at least one of at least one of the group consisting essentially of fabric, padded nylon fabric, and leather.

Even further, it provides such a semi-custom ankle brace system wherein such at least one medial stiff portion is structured and arranged to be placed essentially adjacent an inside (medial) surface of the lower calf; wherein such at least one lateral stiff portion is structured and arranged to be placed essentially adjacent an outside (lateral) surface of the lower calf; wherein such at least one arch stiff portion is structured and arranged to be placed essentially adjacent at least one substantial portion of the arch; and wherein such at least one unitary stiff support further comprises essentially no stiff portions structured and arranged to be placed essentially adjacent a front surface of the lower calf, a rear surface of the lower calf, a top surface of the foot, nor the toes portion of the foot.

Further, it provides such semi-custom ankle brace system, for stabilizing an ankle of a leg, having a lower calf and a foot having an arch and a heel and a toes portion, comprising, in combination: at least one adjustably tightenable portion structured and arranged to essentially surround the lower calf; and at least one other adjustably tightenable portion structured and arranged to essentially tighten around the ankle, the heel, and the arch; wherein such at least one semi-custom ankle brace system is structured and arranged to be worn inside of at least one standard shoe large enough to hold such at least one semi-custom ankle brace system; wherein such at least one semi-custom ankle brace system comprises at least one unitary stiff support structured and arranged to stabilize the ankle in relation to the foot in such at least one semi-custom ankle brace system; and wherein such at least one unitary stiff support consists essentially of at least one low-temperature-thermoplastic material.

Even further, it provides such a semi-custom ankle brace system, wherein such at least one unitary stiff support consists essentially of at least one low-temperature-thermoplastic material comprising substantial malleability at about 180 degrees Fahrenheit. Moreover, it provides such a semi-custom ankle brace system, wherein such unitary stiff support comprises stiff portions comprising at least one medial stiff portion structured and arranged to be placed essentially adjacent an inside (medial) surface of the lower calf, at least one lateral stiff portion structured and arranged to be placed essentially adjacent an outside (lateral) surface of the lower calf, and at least one arch stiff portion structured and arranged to be placed essentially adjacent at least one substantial portion of the arch; wherein such at least one unitary stiff support further comprises essentially no stiff portions structured and arranged to be placed essentially adjacent a front surface of the lower calf, a rear surface of the lower calf, a top surface of the foot, nor the toes portion of the foot. Additionally, it provides such a semi-custom ankle brace system, wherein such at least one unitary stiff support further comprises essentially no heel stiff portion structured and arranged to be placed essentially adjacent to the heel.

In accordance with another preferred embodiment hereof, this invention provides a method of fabricating a semi-custom ankle brace system, in a preset number of sizes, comprising the steps of cutting at least one inner lining from at least one soft flexible material using at least one standard pattern of at least one preset size; attaching such at least one inner lining so that it contains at least one seam essentially parallel to the back of at least one heel from the bottom of the at least one heel to the top of such at least one semi-custom ankle brace; cutting at least one outer lining from at least one soft flexible material using at least one standard pattern of at least one preset size; attaching such at least one outer lining so that it contains at least one seam essentially parallel to the back of at least one heel from the bottom of the at least one heel to the top of such at least one semi-custom ankle brace; cutting at least one unitary stiff support, using at least one standard pattern of at least one preset size, from at least one sheet of at least one thermally formable material; forming such at least one unitary stiff support to the shape of at least one standard sized last; holding such at least one unitary stiff support between such at least one inner lining and such at least one outer lining of such at least one semi-custom ankle brace; attaching at least one perimeter of such at least one inner lining and such at least one outer lining together, so as to encase such at least one unitary stiff support; attaching at least one edge of at least one tongue to at least one lining of such at least one semi-custom ankle brace system; and attaching at least one adjustably tightenable closure in such at least one semi-custom ankle brace system.

Also, it provides such a method, wherein the step of attaching comprises the step of at least one of the group consisting essentially of stitching and gluing. In addition, it provides such a method, wherein the step of attaching at least one closure comprises the step of attaching at least one of the group consisting essentially of hook and loop fastener, elastic, and cinched strap. And, it provides such a method, wherein the step of attaching at least one closure comprises the step of inserting a plurality of eyelet grommets in such at least one ankle brace system; and threading at least one lace through such plurality of eyelet grommets. Further, it provides such a method, further comprising the step of attaching such at least one inner lining and such at least one outer lining together around at least one portion of at least one perimeter of such at least one unitary stiff support. Even further, it provides such a method, wherein the step of attaching comprises the step of at least one of the group consisting essentially of stitching and gluing.

In accordance with another preferred embodiment hereof, this invention provides a method of using a semi-custom ankle brace system comprising the steps of selecting at least one semi-custom ankle brace system of at least one appropriate size to fit at least one foot of at least one patient; placing at least one thermally protective sock on at least one foot of at least one patient; submerging such at least one semi-custom ankle brace system in at least one fluid at at least one temperature and for at least one time sufficient to soften the at least one thermoplastic material of such at least one semi-custom ankle brace system; placing such at least one semi-custom ankle brace system over such at least one thermally protective sock; tightening such at least one semi-custom ankle brace system to the at least one foot of the at least one patient; moving the at least one foot and its bone structure to at least one preferred position and holding the at least one foot until such at least one thermoplastic material of such at least one semi-custom ankle brace system cools; wherein such at least one semi-custom ankle brace system is given at least one semi-custom fit to the at least one patient's at least one foot.

And, it provides such a method, further comprising the step of placing at least one compression sock over such at least one semi-custom ankle brace system to apply compression evenly over the at least one foot. Further, it provides such a method, further comprising the step of placing at least one plastic bag over such at least one semi-custom ankle brace system, prior to placing such at least one compression sock over such at least one semi-custom ankle brace system.

In accordance with another preferred embodiment hereof, this invention provides a method for retail distribution of semi-custom ankle brace systems, to be worn inside shoes, comprising the steps of providing to orthotics retailers at least one inventory listing of such at least one semi-custom ankle brace systems in at least one range of sizes no greater than available shoe sizes; providing to such retailers at least one price at which such retailers may order and purchase such initial selection of such semi-custom ankle brace systems; taking at least one first order from such retailers and providing to such retailers such at least one initial selection of such at least one semi-custom ankle brace systems; and providing at least one training to such retailers in proper application and fitting, using conformation of low-temperature thermoplastic unitary stiff supports of such at least one semi-custom ankle brace systems. Even further, it provides such a method, further comprising providing to such retailers at least one price at which such retailers may re-order and purchase at least one additional stock of such at least one semi-custom ankle brace systems; the method, further comprising taking at least one additional order from such retailers and providing to such retailers such at least one additional stock of such at least one semi-custom ankle brace systems as required to replenish such retailers stocks. Even further, it provides such a method, wherein the step of providing at least one training to such retailers comprises the step of providing at least one in-person training. Even further, it provides such a method, wherein the step of providing at least one training to such retailers comprises the step of providing at least one training material. Even further, it provides such a method, wherein the step of providing at least one training material further comprises the step of providing at least one video training material in the form of at least one of the group consisting essentially of DVD, CD-Rom, and videotape.

In accordance with another preferred embodiment hereof, this invention provides a semi-custom ankle brace system for stabilizing an ankle of a leg, having a lower calf having an inside (medial) surface and an outside (lateral) surface, and having a foot having an arch and a heel and a toes portion, comprising, in combination: unitary stiff support means for stiffly supporting the ankle in relation to the foot; wherein such unitary stiff support means comprises stiff portions comprising at least one medial portion to support the inside (medial) surface of the lower calf, at least one lateral portion to support the outside (lateral) surface of the lower calf, and at least one arch portion to support at least one substantial portion of the arch; wherein such unitary stiff support means further comprises essentially no stiff portions to support front surfaces of the lower calf, rear surfaces of the lower calf, top surfaces of the foot, nor toes portions of the foot. Even further, it provides such a semi-custom ankle brace system, wherein such unitary stiff support means further comprises essentially no stiff portions structured and arranged to support the heel.

Even further, it provides such a semi-custom ankle brace system, further comprising inner lining means for lying between essentially all of such unitary stiff support means and such leg. Even further, it provides such a semi-custom ankle brace system, further comprising outer lining means for lying outside and covering essentially all of such unitary stiff support means. Even further, it provides such a semi-custom ankle brace system, further comprising attachment means for attaching such inner lining means with such outer lining means in such manner as to hold essentially all of such unitary stiff support means within such lining means. Even further, it provides such a semi-custom ankle brace system, further comprising adjustably tightenable closure means for adjustably closing such semi-custom ankle brace system around the ankle. Even further, it provides such a semi-custom ankle brace system, further comprising tongue means for covering at least one gap under the closure means of such semi-custom ankle brace system. Even further, it provides each and every novel feature, element, combination, step and/or method disclosed or suggested by this patent application.

### BRIEF DESCRIPTION OF THE DRAWINGS

FIG. 1 shows a front perspective view of a foot of a patient wearing a semi-custom ankle brace according to the present invention and used by the patient while wearing an essentially normal shoe.

FIG. 1a shows a perspective view illustrating the semi-custom ankle brace system.

FIG. 2 shows a side view of a preferred embodiment of the semi-custom ankle brace of this invention illustrating the location of the thermoplastic unitary stiff supports within the cloth ankle brace.

FIG. 3 shows a three-quarters rear view illustrating a single vertical seam of the boot along the back of the semi-custom ankle brace.

FIG. 4 shows a perspective view illustrating the inside of the boot and how the tongue of the boot attaches to the main portion of the boot.

FIG. 4a shows a perspective view of an alternate embodiment of the semi-custom ankle brace of the current invention.

FIG. 5 shows a top view of how the thermoplastic material is cut prior to preforming around a last.

FIG. 6 shows an illustration of cutting the inner lining during the assembly of the semi-custom ankle brace.

FIG. 6a shows an illustration of cutting the unitary stiff support during the assembly of the semi-custom ankle brace.

FIG. 6b shows an illustration of placing the preformed unitary stiff support around the inner lining during the assembly of the semi-custom ankle brace.

FIG. 6c shows an illustration of cutting the outer lining during the assembly of the semi-custom ankle brace.

FIG. 6d shows an illustration of a fully assembled semi-custom ankle brace.

FIG. 7 shows an illustration of a cut-out thermoplastic unitary stiff support.

FIG. 7a shows an illustration of a side perspective view of the thermoplastic unitary stiff support being pre-formed on a last.

FIG. 7b shows an illustration of a back perspective view of the thermoplastic unitary stiff support being pre-formed on a last.

FIG. 7c shows an illustration of a cooled, pre-formed thermoplastic unitary stiff support.

FIG. 8a shows an illustration of the process of submersing the semi-custom ankle brace in hot water to soften the enclosed unitary stiff support.

FIG. 8b shows an illustration of the step of placing the heated semi-custom ankle brace over the thermally protective sock and wearer's foot.

FIG. 8c shows an illustration of the process of placing the compression sock over the semi-custom ankle brace on the patient's foot.

FIG. 8d shows an illustration of the process of the skilled fitter holding the patient's foot in the corrected position while the unitary stiff support cools.

FIG. 9 shows an illustration of a retail distribution system for semi-custom ankle bracing systems according to a preferred business method of the present invention.

FIG. 10 shows an illustration of a diagram of the steps of fabricating a semi-custom ankle brace according to a preferred embodiment of the present invention.

FIG. 11 shows an illustration of a diagram of the steps of using a semi-custom ankle brace according to a preferred embodiment of the present invention.

### DETAILED DESCRIPTION OF THE BEST MODES AND PREFERRED EMBODIMENTS OF THE INVENTION

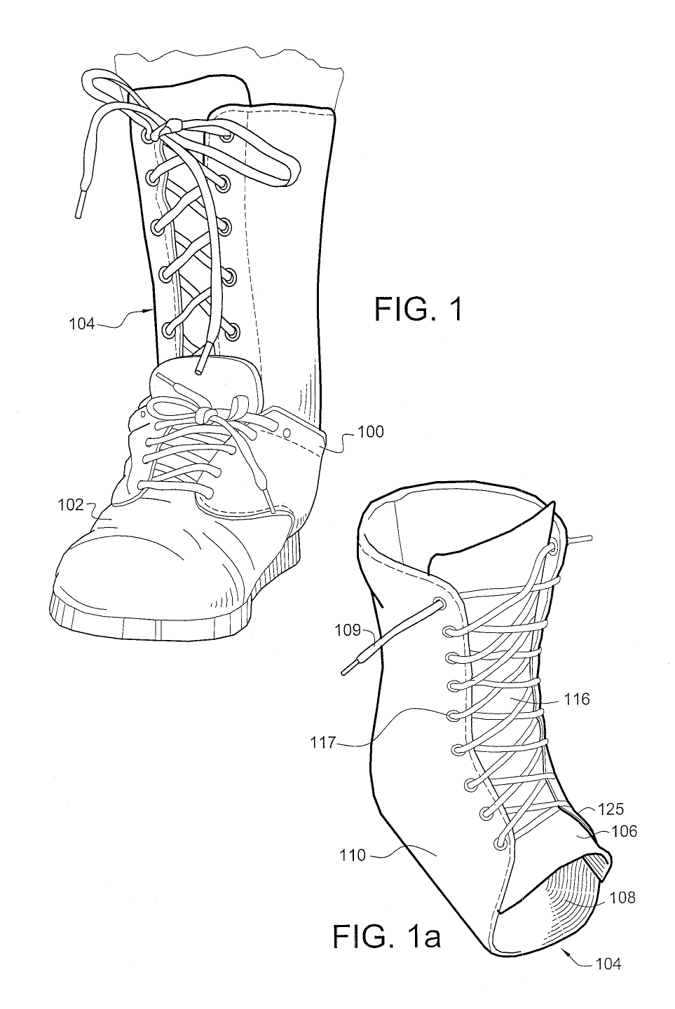

FIG. 1 shows the semi-custom ankle brace 104 of this invention according to a preferred embodiment thereof. As shown, shoe 102 is preferably worn over the semi-custom ankle brace 104, both holding the patient's foot 100. Typically, the semi-custom ankle brace 104 allows for the patient to wear a standard shoe size only ½ size larger than normal. The semi-custom ankle brace 104 provides marked improvement in foot/ankle support for patients with a range of common foot/ankle ailments. Each semi-custom ankle brace 104 is uniquely molded, as hereinafter set out, to meet the specific needs and physical properties of the individual patient's foot 100.

Ailments suggesting the use of the semi-custom ankle brace 104 are ailments such as arthritis, trauma, tendonitis, pronation, inversion, eversion, and bony prominences that would make it difficult for the patient to wear a shoe 102, as illustrated in FIG. 1, and difficult to walk without the use of a brace. Often these patients have had one or more surgeries and may have sensitive scarred areas as well. It is noted for vocabulary purposes that a patient typically has a leg; a lower calf with an inside (medial) surface and an outside (lateral) surface; and a foot having an arch, a heel, and toes.

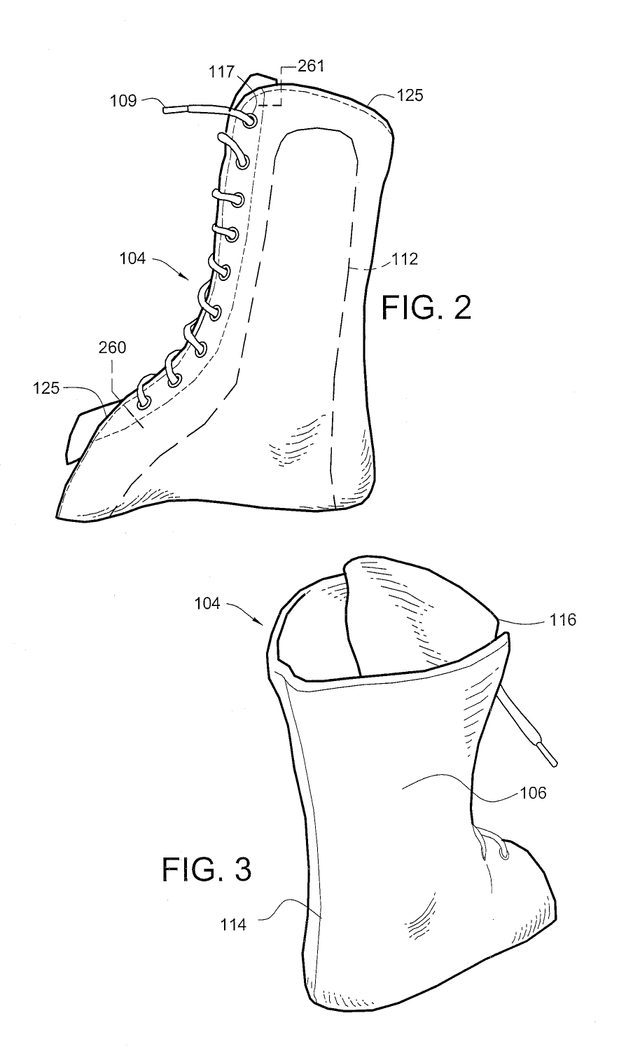

FIG. 1a shows an illustration of the semi-custom ankle brace 104 according to the embodiment of FIG. 1. Preferably, the semi-custom ankle brace 104 comprises a toeless boot 106. Preferably, the toeless boot 106 consists of an inner lining 108 and an outer lining 110, preferably made of soft, flexible materials, and encasing a unitary stiff support 112, as illustrated in FIG. 2. Semi-custom ankle brace 104 further comprises attachment means for attaching the parts of semi-custom ankle brace 104 together, preferably stitches and glue. Semi-custom ankle brace 104 further comprises closure means for adjustably tightening semi-custom ankle brace 104 around the lower calf, ankle, heel, and arch, such as, for example, eyelets and laces, hooks and loops, elastic closures, and cinched straps.

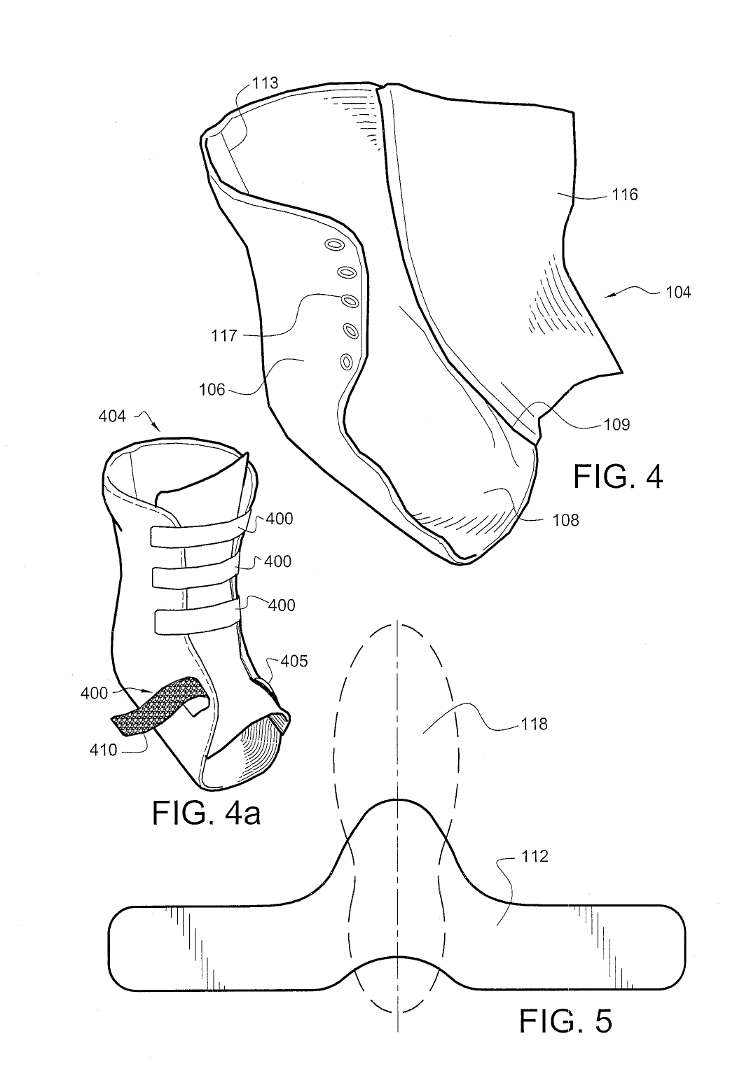

Preferably, semi-custom ankle brace 104 consists of three individual layers: the inner lining 108, the unitary stiff support 112, and the outer lining 110. The various layers comprising the semi-custom ankle brace 104 are made so as to be conformable to the contours of each patient's foot. Preferably, inner lining 108 and outer lining 110 are both arranged to cover the ankle, lower calf, upper foot, and mid-foot, with an opening being left down the entire front, as shown in FIG. 1a. Preferably, inner lining 108 lies between essentially all of unitary stiff support 112 and the patient's leg, as shown in FIG. 4 (embodying herein inner lining means for lying between essentially all of said unitary stiff support means and said leg.)

Preferably, outer lining 110 lies outside of, and covers essentially all of, unitary stiff support 112, as shown in FIG. 1a (embodying herein outer lining means for lying outside and covering essentially all of said unitary stiff support means). Preferably, the portion of semi-custom ankle brace 104 that lies above the top of the foot is structured to be adjustably tightenable around the lower calf. Preferably, the portion of semi-custom ankle brace 104 that lies below the top of the foot is structured to be adjustably tightenable around the heel, ankle, and arch.

The tongue 116 is preferably made from the same flexible material as the rest of the toeless boot 106 of the semi-custom brace 104, as shown. Preferably, tongue 116 covers the gap in the front of semi-custom ankle brace 104, under the closure means, protecting the patient from chafing against the closure means (embodying herein tongue means for covering the gap under the closure means of said semi-custom ankle brace system). Upon reading the teachings of this specification, those skilled in the art will now understand that, under appropriate circumstances, considering such issues as, for example, available materials, user preference, cost, technology, etc., other soft, flexible materials substantially impervious to water damage, such as, for example, fabric, cotton, polyester, felt, woven fabric, knitted fabric, non-woven fabric, leather, 2.5 oz chromium tanned cowhide leather, etc., may suffice for the inner lining, outer lining, and tongue.

Preferably, unitary stiff support 112 is used to provide substantially rigid support for the patient's foot 100, and to maintain the patient's sole in a relatively horizontal plane when the lower leg is vertical, thus assisting the patient to walk as normally as possible. Preferably, unitary stiff support 112 consists of essentially three portions: a support portion 710 placed essentially adjacent to and supporting the medial (inside) surface of the lower calf; a support portion 700 placed essentially adjacent to and supporting the lateral (outside) surface of the lower calf; and a support portion 715 placed essentially adjacent to and supporting a substantial portion of the arch of the foot. These portions permit unitary stiff support 112, in combination with toeless boot 106, to provide medial and lateral support to the ankle. Preferably, no stiff support portions are provided to be placed adjacent to or support: the front or back surfaces of the lower calf; the top surface of the foot; or the toes portion of the foot. Alternatively, an additional stiff support portion of unitary stiff support 112 may be provided to be placed adjacent to and support the heel (embodying herein a semi-custom ankle brace system for stabilizing an ankle of a leg, having a lower calf having an inside (medial) surface and an outside (lateral) surface, and having a foot having an arch and a heel and a toes portion, comprising, in combination: unitary stiff support means for stiffly supporting the ankle in relation to the foot; wherein said unitary stiff support means comprises stiff portions comprising at least one medial portion to support the inside (medial) surface of the lower calf, at least one lateral portion to support the outside (lateral) surface of the lower calf, and at least one arch portion to support at least one substantial portion of the arch; wherein said unitary stiff support means further comprises essentially no stiff portions to support front surfaces of the lower calf, rear surfaces of the lower calf, top surfaces of the foot, nor toes portions of the foot; and also embodying herein wherein said unitary stiff support means further comprises essentially no stiff portions structured and arranged to support the heel).

Preferably, the adjustably tightenable closure is structured to adjustably tighten the semi-custom ankle brace 104 around the ankle. The adjustably tightenable closure is preferably structured to close the front of semi-custom ankle brace 104 between point 260 and point 261, as shown in FIG. 2. In a preferred embodiment, the closure comprises eyelet grommets 117 and laces 109 as illustrated in FIG. 2 (embodying herein adjustably tightenable closure means for adjustably close said semi-custom ankle brace system around the ankle). FIG. 4a shows an illustration of an alternate preferred embodiment wherein the closure may be hook and loop fasteners 400. Upon reading the teachings of this specification, those skilled in the art will now understand that, under appropriate circumstances, considering such issues as cost, user preference, available materials, technology, other methods of adjustably closing semi-custom ankle brace 104, such as hook and loop fasteners, elastic closures, cinched straps, etc., may suffice.

The attachment means, comprising attachers, are preferably structured to attach the various parts of semi-custom ankle brace 104 to each other. For example, attachers preferably join the linings 108 and 110 to each other and join the tongue 116 to the linings 108 and 110 and join the closure means to the toeless boot 106 and join the back seams 113 and 114 of the linings 108 and 110. Attachers may comprise stitches, glue, melt welding, or other appropriate means for joining flexible materials together (embodying herein attachment means for attaching said inner lining means with said outer lining means in such manner as to hold essentially all of said unitary stiff support means within said lining means). Upon reading the teachings of this specification, those skilled in the art will now understand that, under appropriate circumstances, considering such issues as materials requirements, user preference, technology, cost, etc., other attachment means, such as glue, melt welding, edge binding, etc., may suffice.

FIG. 2 shows a side view illustrating the preferred location of the unitary stiff support 112 within the semi-custom ankle brace 104. Unitary stiff support 112 preferably is held between inner lining 108 and outer lining 110. Preferably, unitary stiff support 112 is fabricated from plastic, thermoplastic, low-temperature thermoplastic, or low-temperature thermoplastic comprising substantial malleability at about 180 degrees Fahrenheit. Most preferably, the unitary stiff support 112 is fabricated from ORTHOPLAST® thermoplastic, manufactured by Johnson and Johnson. Preferably, unitary stiff support 112 is fabricated from a sheet of one-eighth-inch-thick thermoplastic. Upon reading the teachings of this specification, those skilled in the art will now understand that, under appropriate circumstances, considering such issues as available materials, cost, user preference, technology, etc., other low temperature thermally formable materials, such as MULTIFORM PLASTIC™, X-LITE PLUS™, EZEFORM SPLINTING MATERIAL™, MULTIFORM CLEAR™, etc., may suffice.

FIG. 3 shows a three-quarters rear view illustrating a single vertical seam 114 of the toeless boot 106 according to the preferred embodiment of FIG. 1. Preferably, the seam 114 follows the back of the heel in a vertical fashion, up the back of the toeless boot 106, as shown. Upon reading the teachings of this specification, those skilled in the art will now understand that, under appropriate circumstances, considering such issues as wide feet, user preference, cost, technology, etc., other seam placements and quantities, such as two seams going up opposite sides of the heel, two seams going from the heel toward the top of the foot, three seams, etc., may suffice.

FIG. 4 shows a perspective view illustrating the inner lining 108, and also illustrating how the tongue 116 of the toeless boot 106 preferably attaches to the front edge 109 of the toeless boot 106. FIG. 4 also shows an illustration of a single vertical seam 113 of the toeless boot 106 according to the preferred embodiment of FIG. 1. Preferably, the seam 113 follows the back of the heel in a vertical fashion, up the inside back of the toeless boot 106, as shown. Upon reading the teachings of this specification, those skilled in the art will now understand that, under appropriate circumstances, considering such issues as wide feet, user preference, cost, technology, etc., other seam placements and quantities, such as two seams going up opposite sides of the heel, two seams going from the heel toward the top of the foot, three seams, etc., may suffice.

FIG. 4a shows an illustration of an alternate preferred embodiment wherein the closure may be hook and loop fasteners 400. One or more hook and loop fasteners 400 may be used. Preferably, hook and loop fasteners 400 comprise at lease one loop portion 405 and at least one hook portion 410. Upon reading the teachings of this specification, those skilled in the art will now understand that, under appropriate circumstances, considering such issues as available materials, user preference, technology, cost, etc., other hook and loop fastener arrangements, such as reversing the positions of the hook portion and the loop portion, wider straps, one wide strap, etc., may suffice.

FIG. 5 shows a top view showing how the thermoplastic material of unitary stiff support 112 is preferably cut prior to performing and assembling within the toeless boot 106. Preferably, the unitary stiff support 112 is cut from a flat sheet of the thermoplastic material. Most preferably, the unitary stiff support 112 is preformed using low heat and a last 118 prior to holding unitary stiff support 112 in the layers of the toeless boot 106. Most preferably the unitary stiff support 112 is molded to its final shape when heated and conformed to the patient's foot 100, as is further described below.

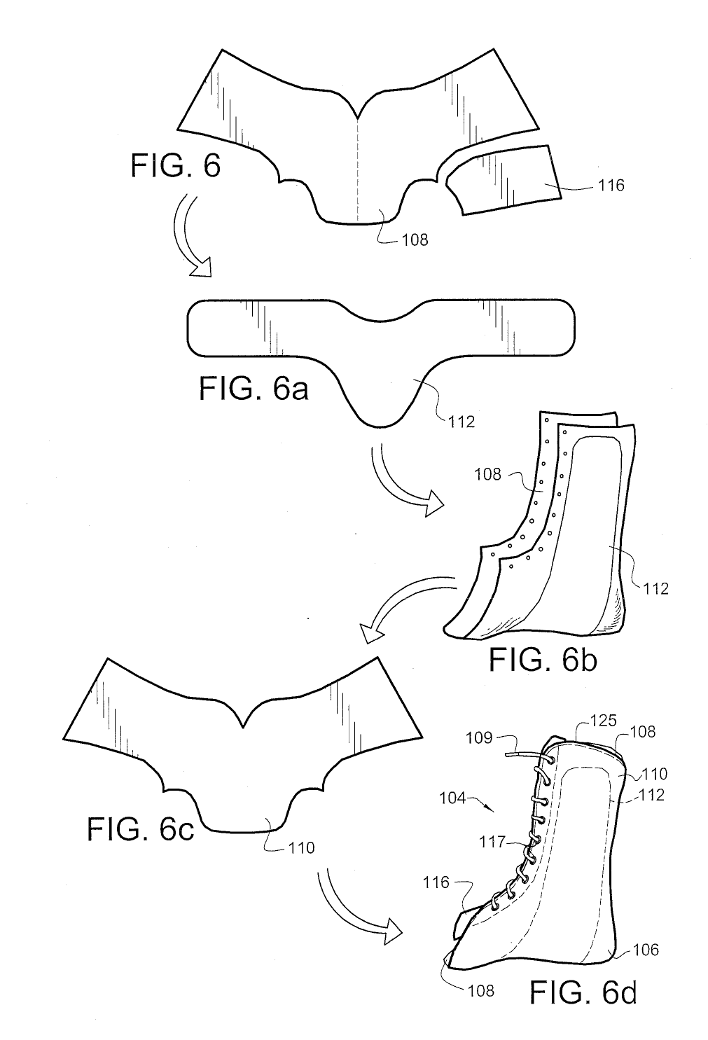

FIGS. 6, 6a, 6b, 6c, and 6d illustrate a preferred method of making the semi-custom ankle brace 104. Preferably, the first step in making a semi-custom ankle brace 104 is cutting an inner lining 108 and a tongue 116 (as shown in FIG. 6) and an outer lining 110 (as shown in FIG. 6c) from a soft flexible material, preferably using a standard pattern. Such standard patterns may conform to various preset sizes, the selection of which is based on customer demands, for example, XS, S, M, L, and XL. In the preferred embodiment, the inner lining 108, outer lining 110, and tongue 116 preferably consist of a soft flexible material substantially impervious to water damage, most preferably a soft padded-nylon fabric. Specifically shown in FIG. 6d is a perspective view of the preferred semi-custom ankle brace 104, having an outer lining 110 substantially covering a pre-determined length of the patient's calf, a substantial portion of the patient's sole, and/or the patient's heel.

FIG. 6d shows an illustration of the preferred cut-out shape of a unitary stiff support 112, which in the preferred embodiment is made of a thermoplastic such as ORTHOPLAST®. Preferably, unitary stiff support is then pre-formed on a last 118. Preferably, in the next step, the inner lining 108 is attached, preferably stitched, along the posterior of the semi-custom ankle brace 104 extending from the patient's heel upward to the top of the semi-custom ankle brace 104, forming seam 113 (as in the example shown in FIG. 4). The outer lining 110 is also attached, preferably stitched, along the posterior of the semi-custom ankle brace 104 extending from the patient's heel upward to the top of the semi-custom ankle brace 104, forming seam 114 (as in the example shown in FIG. 3). FIG. 6b shows an illustration of pre-formed unitary stiff support 112 being placed in its preferred proper position relative to inner lining 108.

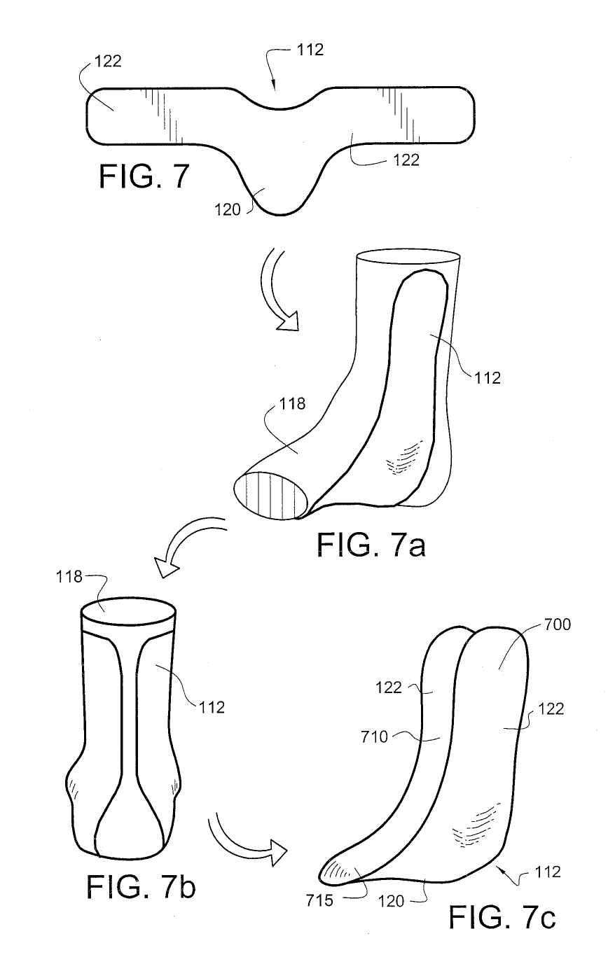

FIG. 7-7c show illustrations of more details about preferred pre-forming of the unitary stiff support 112 for use within the material layers in making the semi-custom ankle brace 112.

FIG. 7 shows the approximate shape of unitary stiff support 112, which is preferably cut with dimensions of approximately 16″ by 24″ from a sheet of heat formable material. In the preferred embodiment, an ORTHOPLAST® thermoplastic material is used.

FIG. 7a shows an illustration of the next step of fabrication, wherein the unitary stiff support 112 is thermally formed to the shape of a standard sized last 118, the selection of which is based on customer demands.

FIG. 7b shows an illustration of a back view of the pre-formed unitary stiff support 112 on the last 118.

FIG. 7c shows an illustration of that cooled, pre-formed unitary stiff support 112 maintains its shape after being removed from last 118.

After the pre-forming step, the pre-formed unitary stiff support 112 is preferably placed and held between the inner and outer linings of the semi-custom ankle brace 104, as is shown in FIGS. 6b and 6d. Preferably, the outer lining 110 is then attached to the inner lining 108 along the perimeter 125 of the inner lining 108 and the outer lining 110, thereby forming the toeless boot 106, which holds and contains unitary stiff support 112. Also, preferably, the inner lining 108 and outer lining 110 may be attached together along at least part of the perimeter of unitary stiff support 112, illustrated by the dashed lines, which also indicate unitary stiff support 112 in FIG. 6d, in order to prevent unitary stiff support 112 from sliding within semi-custom ankle brace 104.

Preferably, next, the tongue 116 may be attached to the edge 109 of semi-custom ankle brace 104, as shown in FIG. 4. Tongue 116 may be attached to either edge of semi-custom ankle brace 104, as desired. Preferably, the last step in the making of the semi-custom ankle brace 104 is the attachment of the adjustably tightenable closure. One preferred embodiment of a closure is laces and grommets. FIG. 1a shows the laces 109 and grommets 117 along the front centerline of the foot; however, the placement of the closure may be altered to provide a more comfortable fit to the patient when required without detracting from the present invention. The final fitting and adjustments, if needed, will be as determined by the skill and experience of the brace maker or fitter. Alternatively, an opening may be made in toeless boot 106, in order to permit unitary stiff support 112 to be temporarily removed by the patient, in order to permit easy laundering of toeless boot 106. For example, an opening may be made along the perimeter 125 of toeless boot 106 at either the front sole portion, at the top of the back of the calf, or in the back seam 114 of outer lining 110. An opening also permits an alternate method of construction, wherein toeless boot 106 is partially or completely constructed prior to insertion of preformed unitary stiff support 112. An opening may also comprise a closure, such as, for example, hook and loop fasteners, a zipper, or a button.

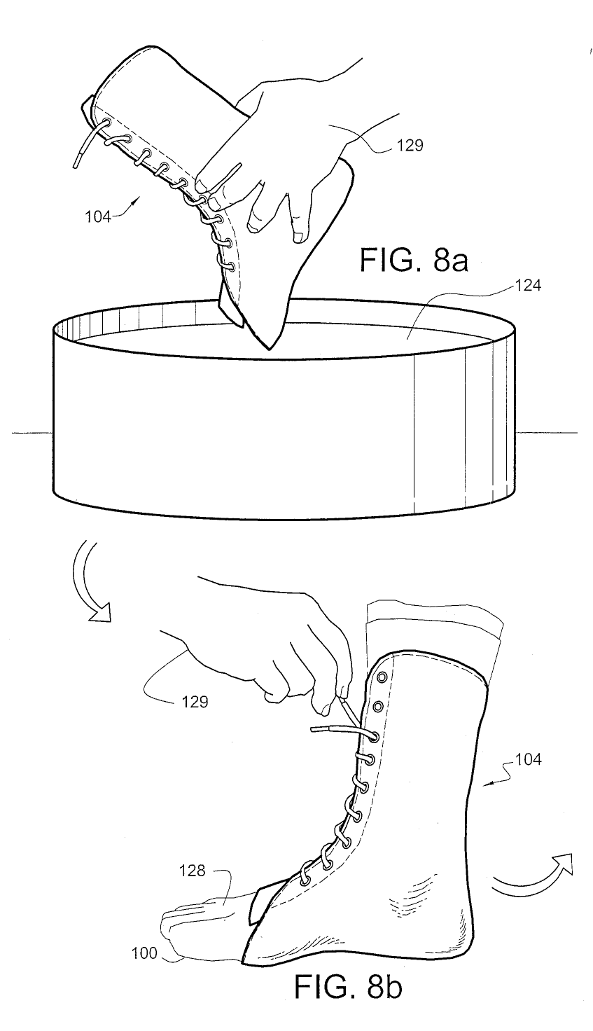

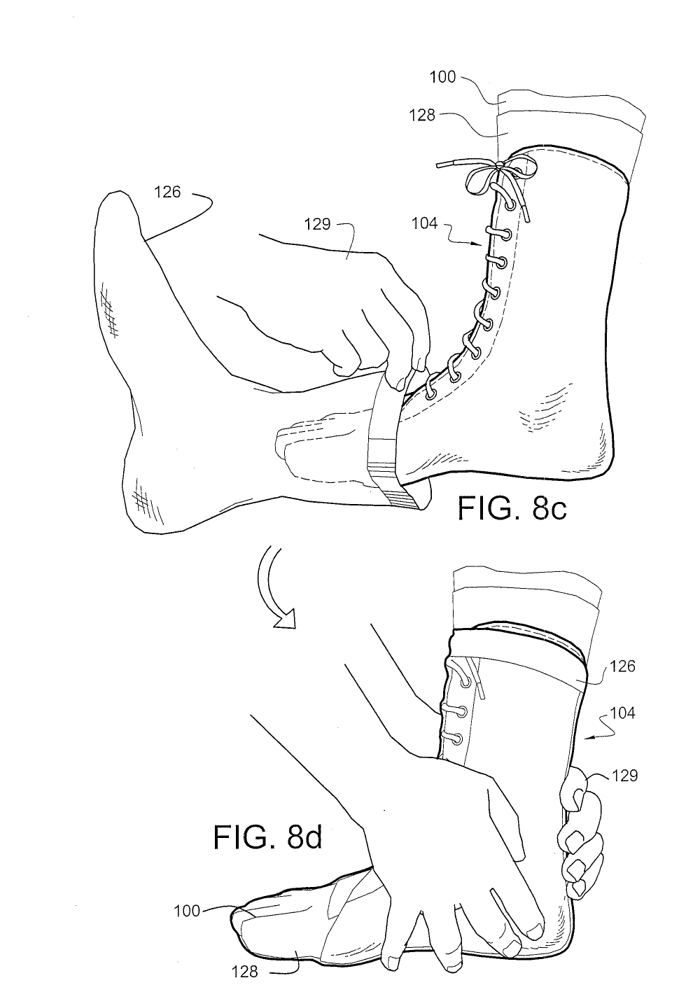

FIG. 8a shows an illustration of the preferred method of fitting the semi-custom ankle brace 104. Prior to the initial semi-custom ankle brace 104 fitting process, a person skilled in the art of foot ailments and brace making (the fitter 129) performs an examination of the patient's foot 100. A determination of the closest size semi-custom ankle brace system 104 needed by the patient is made. The appropriate size of semi-custom ankle brace system 104 is then selected, ordered, and received by the retailer. Preferably, next, the fitter 129 places a thermally protective sock 128 over the foot and lower leg of the patient.

Preferably, as shown in FIG. 8a, the semi-custom ankle brace 104 is then heated in hot water 124 to a temperature, and for a time, sufficient to make unitary stiff support 112 within semi-custom ankle brace 104 substantially malleable. Preferably, when unitary stiff support 112 is made of approximately one-eighth-inch thick ORTHOPLAST®, semi-custom ankle brace 104 is preferably heated in hot water 124 at approximately 180 degrees Fahrenheit for preferably approximately 2-3 minutes, unitary stiff support 112 is substantially malleable so that one of ordinary skill in the art can conveniently deform and shape unitary stiff support 112 with their hands (heat protective gloves may be needed). Upon reading the teachings of this specification, those skilled in the art will now understand that, under appropriate circumstances, considering such issues as materials requirements, user preference, technology, conditions of end use, cost, etc., other approximate temperatures, such as 120 degrees Fahrenheit, 160 degrees Fahrenheit, 212 degrees Fahrenheit, etc., may suffice.

FIG. 8b shows the preferred next step of use, wherein semi-custom ankle brace 104 is placed over the thermally protective sock 128 while unitary stiff support 112 is still substantially malleable. Preferably, semi-custom ankle brace 104 is then tightened and adjusted, by use of the closure, to provide an optimal fit to the patient.

FIG. 8c shows an illustration of an optional use step wherein a fitter 129 may apply a compression sock 126 over semi-custom ankle brace 104 in order to further conform semi-custom ankle brace 104 to the patient. In another optional step, when a compression sock 126 is used, a plastic bag (not shown) may be placed over semi-custom ankle brace 104 prior to placing compression sock 126 over semi-custom ankle brace 104. The plastic bag, which is slippery, makes it easier to apply compression sock 126.

FIG. 8d shows an illustration of the next preferred step of fabrication, wherein fitter 129 adjusts the position of the patient's foot 100 to the clinically proper position. Preferably, the compression sock 126 (or fitter 129, by hand) then uniformly conforms the shape of the unitary stiff support 112 to the patient's foot 100 while it cools, as shown. Once the unitary stiff support has cooled and hardened, a semi-custom fit of the semi-custom ankle brace 112 has been achieved. Alternatively, where toeless boot 106 has an opening, the foregoing steps may be used to shape unitary stiff support 112 to the patient's foot 100 prior to inserting unitary stiff support 112 into toeless boot 106.

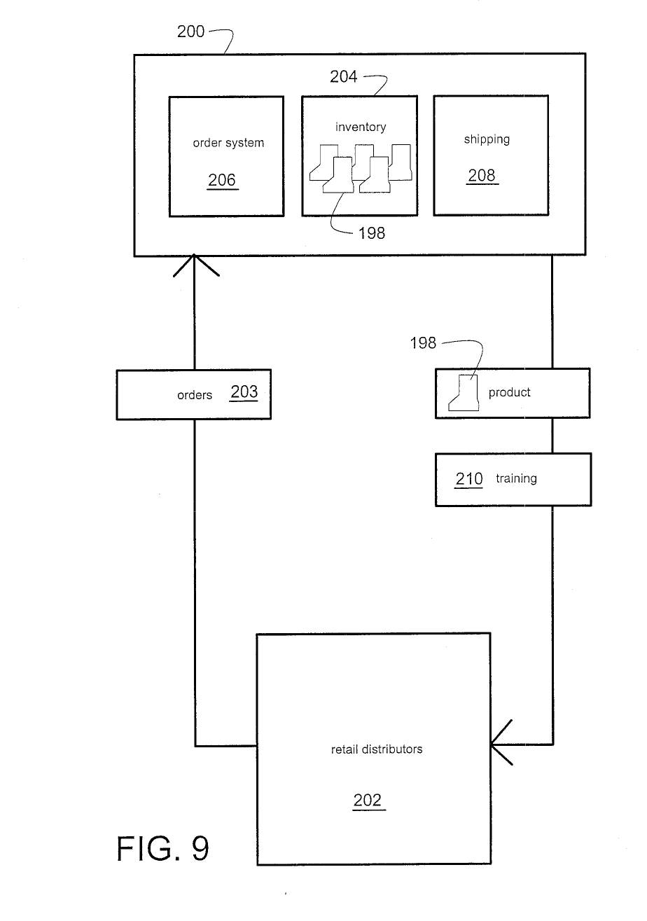

FIG. 9 shows an illustration of a retail distribution system for semi-custom ankle brace systems 198 according to a preferred business method of the present invention. Preferably, the semi-custom ankle bracing systems 198 are sold through a network of orthotics retail distributors 202. A central distribution center 200 preferably provides a ready-to-ship inventory 204, a customer order system 206, and a packaging/shipping component 208, as shown. Preferably, the distribution center 200 provides the retail distributors 202 an inventory listing of the available semi-custom ankle bracing systems 198. The inventory listing may be published via the Internet or by printed catalog.

Preferably, a distribution representative assists new retailers in making an initial selection of the semi-custom ankle bracing systems 198 necessary for start-up retail sales. Preferably, pricing to the retail distributors 202 is established through the distribution representative or through published lists. Retail distributors 202 order 203 and are preferably sold an initial stock of semi-custom ankle bracing systems. Preferably, training 210 is provided to instruct retailers in the proper application and fitting of the semi-custom ankle bracing systems 198 using thermoplastic molding methods. Preferably, training is self directed, provided on media such as compact disks, DVDs, or videotape. Alternatively, on-site and/or remote location instruction may be used. Preferably, subsequent to initial start-up sales, retail distributors 202 re-order and purchase additional stock of the semi-custom ankle bracing systems 198 at a price to replenish depleted stocks. Thus, preferably, the steps for retail distribution of semi-custom ankle brace systems comprise the following steps.

A first step is to provide an inventory listing of semi-custom ankle brace systems, in a range of sizes, to an orthotics retailer (embodying herein the step of providing to orthotics retailers at least one inventory listing of such at least one semi-custom ankle brace systems in at least one range of sizes no greater than available shoe sizes). A second step is to provide a price for the inventory to an orthotics retailer (embodying herein the step of providing to such retailers at least one price at which such retailers may order and purchase such initial selection of such semi-custom ankle brace systems). A third step is to take a first order for semi-custom ankle brace systems from the orthotics retailer and supply semi-custom ankle brace systems to the retailers (embodying herein the step of taking at least one first order from such retailers and providing to such retailers such at least one initial selection of such at least one semi-custom ankle brace systems). A fourth step is to provide training in fitting the semi-custom ankle brace systems to the orthotics retailer (embodying herein the step of providing at least one training to such retailers in proper application and fitting, using conformation of low-temperature thermoplastic unitary stiff supports of such at least one semi-custom ankle brace systems).

The provided training may be in-person training (embodying herein the step of providing at least one in-person training) or training materials (embodying herein the step of providing at least one training material). The training materials may be videos provided on DVDs, CD-ROMs, or videotape (embodying herein the step of providing at least one video training material in the form of at least one of the group consisting essentially of DVD, CD-Rom, and videotape). An optional fifth step is to provide orthotics retailers with a price for re-ordering semi-custom ankle braces (embodying herein the step of providing to such retailers at least one price at which such retailers may re-order and purchase at least one additional stock of such at least one semi-custom ankle brace systems).

An optional seventh step is to take another order for semi-custom ankle brace systems from the orthotics retailer and supply additional semi-custom ankle brace systems to the retailers (embodying herein the step of taking at least one additional order from such retailers and providing to such retailers such at least one additional stock of such at least one semi-custom ankle brace systems as required to replenish such retailers stocks).

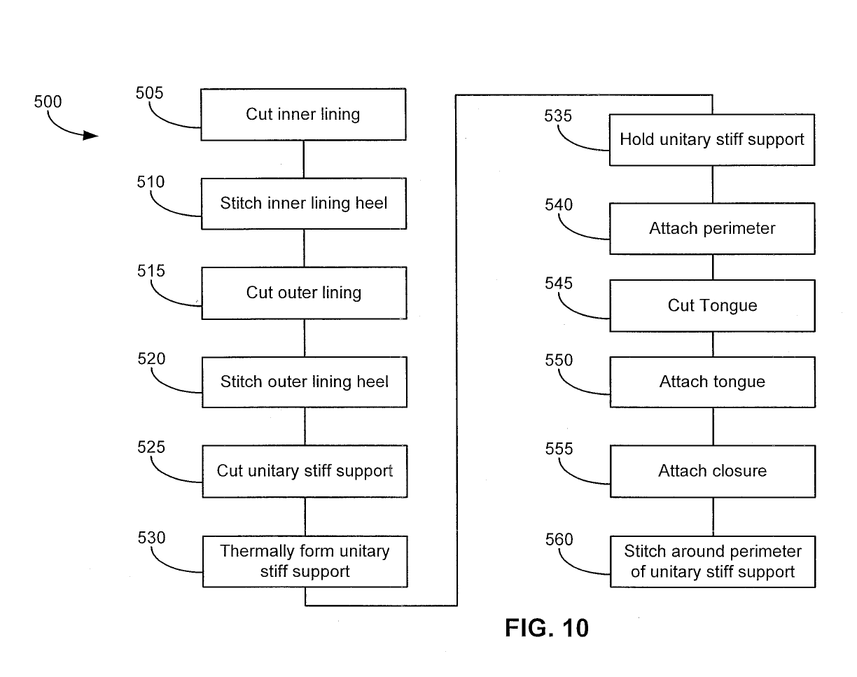

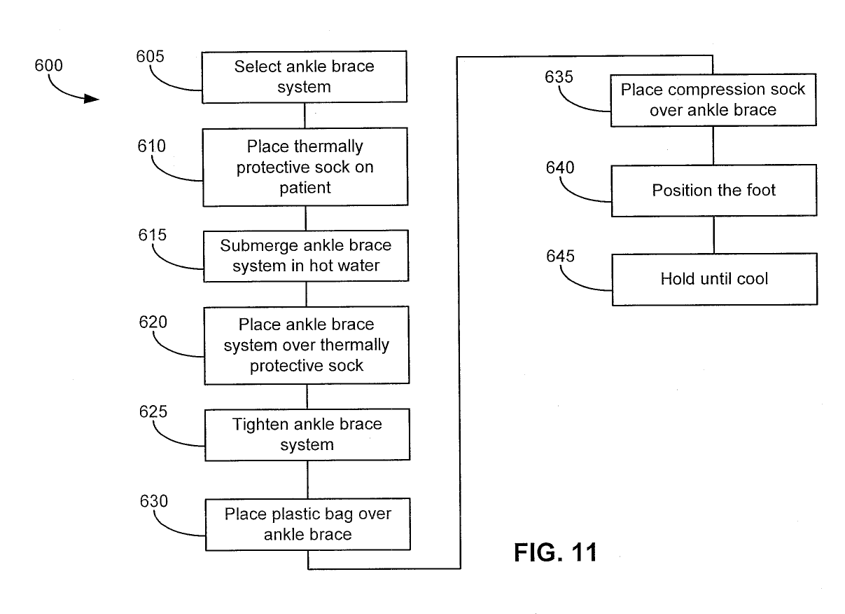

FIG. 10 shows an illustration of a diagram of an example of the steps of a preferred method of fabricating 500 a semi-custom ankle brace 104 according to the present invention. Preferably, a first step is to cut 505 an inner lining 108 from a soft flexible material using a pattern of a preset size (embodying herein cutting at least one inner lining from at least one soft flexible material using at least one standard pattern of at least one preset size). Preferably, a second step is to attach 510 the heel seam 113 of the inner lining 108 from the bottom of the heel to the top of the semi-custom ankle brace 104 (embodying herein attaching such at least one inner lining so that it contains at least one seam essentially parallel to the back of at least one heel from the bottom of the at least one heel to the top of such at least one semi-custom ankle brace). Preferably, a third step is to cut 515 an outer lining 110 from a soft flexible material using a pattern of a preset size (embodying herein cutting at least one outer lining from at least one soft flexible material using at least one standard pattern of at least one preset size). Preferably, a fourth step is to attach 520 the heel seam 114 of the outer lining 110 from the bottom of the heel to the top of the semi-custom ankle brace 104 (embodying herein attaching such at least one outer lining so that it contains at least one seam essentially parallel to the back of at least one heel from the bottom of the at least one heel to the top of such at least one semi-custom ankle brace). Preferably, a fifth step is to cut 525 a unitary stiff support 112 from a thermally formable material using a pattern of a preset size (embodying herein cutting at least one unitary stiff support, using at least one standard pattern of at least one preset size, from at least one sheet of at least one thermally formable material).

Preferably, a sixth step is to form 530 the unitary stiff support 112 to the shape of a standard sized last (embodying herein forming such at least one unitary stiff support to the shape of at least one standard sized last). Preferably, a seventh step is to hold 535 the unitary stiff support 112 within the inner 108 and outer 110 linings (embodying herein holding such at least one unitary stiff support between such at least one inner lining and such at least one outer lining of such at least one semi-custom ankle brace). Preferably, an eighth step is to attach a perimeter 125 of the inner 108 and outer 110 linings together, forming toeless boot 106 (embodying herein attaching at least one perimeter of such at least one inner lining and such at least one outer lining together, so as to encase such at least one unitary stiff support).

Preferably, a ninth step is to cut 545 a tongue 116 from a soft flexible material using a pattern of a preset size (embodying herein cutting at least one outer lining from at least one soft flexible material using at least one standard pattern of at least one preset size). Preferably, a tenth step is to attach 550 a tongue 116 to the side 109 of the toeless boot 106 (embodying herein attaching at least one edge of at least one tongue to at least one lining of such at least one semi-custom ankle brace system). Preferably, an eleventh step is to attach 555 an adjustably tightenable closure to toeless boot 106, thereby forming semi-custom ankle brace 104 (embodying herein attaching at least one adjustably tightenable closure in such at least one semi-custom ankle brace system). An optional eleventh step is to attach 560 the inner and outer linings of semi-custom ankle brace 104 together around the perimeter of the unitary stiff support 112, forming a pocket to prevent unitary stiff support 112 from sliding inside semi-custom ankle brace 104 (embodying herein the step of attaching such at least one inner lining and such at least one outer lining together around at least one portion of at least one perimeter of such at least one unitary stiff support).

FIG. 11 shows an illustration of a diagram of an example of the preferred steps of a method of using 600 a semi-custom ankle brace 104 according to the present invention. A first preferred step is select 605 a semi-custom ankle brace system 104 of an appropriate size for the patient (embodying herein the step of selecting at least one semi-custom ankle brace system of at least one appropriate size to fit at least one foot of at least one patient). A second preferred step is to place 610 a thermally protective sock 128 on the patient's foot (embodying herein the step of placing at least one thermally protective sock on at least one foot of at least one patient). A third preferred step is to submerge 615 the semi-custom ankle brace 104 in hot water 124 until unitary stiff support 112 is substantially malleable (embodying herein the step of submerging such at least one semi-custom ankle brace system in at least one fluid at at least one temperature and for at least one time sufficient to soften the at least one thermoplastic material of such at least one semi-custom ankle brace system).

A fourth preferred step is to place 620 the semi-custom ankle brace 112 over the thermally protective sock 128 on the patient (embodying herein the step of placing such at least one semi-custom ankle brace system over such at least one thermally protective sock). A fifth preferred step is to tighten 625 the semi-custom ankle brace system 104 until a clinically desired tension is achieved (embodying herein the step of tightening such at least one semi-custom ankle brace system to the at least one foot of the at least one patient). An optional sixth step is to place 620 a plastic bag (not shown) over the semi-custom ankle brace 104 (embodying herein the step of placing at least one plastic bag over such at least one semi-custom ankle brace system, prior to placing such at least one compression sock over such at least one semi-custom ankle brace system). A seventh preferred step is to place 635 a compression sock 128 over the semi-custom ankle brace 104 (embodying herein the step of placing at least one compression sock over such at least one semi-custom ankle brace system to apply compression evenly over the at least one foot).

An eighth preferred step is to position 640 the foot 100 in a clinically desirable position while the thermoplastic cools (embodying herein the step of moving the at least one foot and its bone structure to at least one preferred position and holding the at least one foot until such at least one thermoplastic material of such at least one semi-custom ankle brace system cools). A ninth preferred step is to hold the foot 100 in a clinically desirable position until unitary stiff support 112 has cooled sufficiently to no longer be substantially malleable (embodying herein the step of wherein such at least one semi-custom ankle brace system is given at least one semi-custom fit to the at least one patient's at least one foot).

Although applicant has described applicant's preferred embodiments of this invention, it will be understood that the broadest scope of this invention includes modifications such as diverse shapes, sizes, and materials. Such scope is limited only by the below claims as read in connection with the above specification. Further, many other advantages of applicant's invention will be apparent to those skilled in the art from the above descriptions and the below claims.

What is claimed is:

1. A method of fabricating at least one semi-custom ankle brace in a preset number of sizes, comprising the steps of:
a) cutting at least one inner lining from at least one soft flexible material using at least one standard pattern of at least one preset size;
b) attaching such at least one inner lining so that it contains at least one seam essentially parallel to the back of at least one heel from the bottom of the at least one heel to the top of such at least one semi-custom ankle brace;
c) cutting at least one outer lining from at least one soft flexible material using at least one standard pattern of at least one preset size;
d) attaching such at least one outer lining so that it contains at least one seam essentially parallel to the back of at least one heel from the bottom of the at least one heel to the top of such at least one semi-custom ankle brace;
e) cutting at least one unitary stiff support, using at least one standard pattern of at least one preset size, from at least one sheet of at least one thermally formable material;
f) forming such at least one unitary stiff support to the shape of at least one standard sized last;
g) holding such at least one unitary stiff support between such at least one inner lining and such at least one outer lining of such at least one semi-custom ankle brace;
h) attaching at least one perimeter of such at least one inner lining and such at least one outer lining together, so as to encase such at least one unitary stiff support;
i) attaching at least one edge of at least one tongue to at least one lining of such at least one semi-custom ankle brace system; and
j) attaching at least one adjustably tightenable closure in such at least one semi-custom ankle brace system.

2. The method according to claim 1, wherein the step of attaching comprises the step of at least one of the group consisting essentially of
a) stitching and
b) gluing.

3. The method according to claim 1, wherein the step of attaching at least one closure comprises the step of attaching at least one of the group consisting essentially of
a) hook and loop fastener,
b) elastic, and
c) cinched strap.

4. The method according to claim 1, wherein the step of attaching at least one closure comprises
a) inserting a plurality of eyelet grommets in such at least one semi-custom ankle brace; and
b) threading at least one lace through such plurality of eyelet grommets.

5. The method according to claim 1, further comprising the step of attaching such at least one inner lining and such at least one outer lining together around at least one portion of at least one perimeter of such at least one unitary stiff support.

6. The method according to claim 5, wherein the step of attaching comprises the step of at least one of the group consisting essentially of
a) stitching and
b) gluing.

7. A method of using a semi-custom ankle brace system, comprising the steps of:
a) selecting at least one semi-custom ankle brace system of at least one appropriate size to fit at least one foot of at least one patient;
b) placing at least one thermally protective sock on at least one foot of at least one patient;
c) submerging such at least one semi-custom ankle brace system in at least one fluid at at least one temperature and for at least one time sufficient to soften the at least one thermoplastic material of such at least one semi-custom ankle brace system;
d) placing such at least one semi-custom ankle brace system over such at least one thermally protective sock;
e) tightening such at least one semi-custom ankle brace system to the at least one foot of the at least one patient;
f) moving the at least one foot and its bone structure to at least one preferred position and holding the at least one foot until such at least one thermoplastic material of such at least one semi-custom ankle brace system cools;
g) wherein such at least one semi-custom ankle brace system is given at least one semi-custom fit to the at least one patient's at least one foot.

8. The method according to claim 7, further comprising the step of placing at least one compression sock over such at least one semi-custom ankle brace system to apply compression evenly over the at least one foot.

9. The method according to claim 7, further comprising the step of placing at least one plastic bag over such at least one semi-custom ankle brace system, prior to placing such at least one compression sock over such at least one semi-custom ankle brace system.

10. A method used to retail semi-custom ankle brace systems worn inside shoes, comprising the steps of:
a) providing to orthotics retailers at least one inventory listing of such at least one semi-custom ankle brace systems in at least one range of sizes no greater than available shoe sizes;
b) providing to such retailers at least one price at which such retailers may order and purchase such initial selection of such semi-custom ankle brace systems;
c) taking at least one first order from such retailers and providing to such retailers such at least one initial selection of such at least one semi-custom ankle brace systems; and
d) providing at least one training to such retailers in proper application and fitting, using conformation of low-temperature thermoplastic unitary stiff supports of such at least one semi-custom ankle brace systems.

11. The method according to claim 10, further comprising providing to such retailers at least one price at which such retailers may re-order and purchase at least one additional stock of such at least one semi-custom ankle brace systems.

12. The method according to claim 10, further comprising taking at least one additional order from such retailers and providing to such retailers such at least one additional stock of such at least one semi-custom ankle brace systems as required to replenish such retailers stocks.

13. The method according to claim 10, wherein the step of providing at least one training to such retailers comprises the step of providing at least one in-person training.

14. The method according to claim 10, wherein the step of providing at least one training to such retailers comprises the step of providing at least one training material.

15. The method according to claim 14, wherein the step of providing at least one training material further comprises the step of providing at least one video training material in the form of at least one of the group consisting essentially of
a) DVD,
b) CD-Rom, and
c) videotape.

16. A system used to stabilize an ankle of a leg, having a lower calf having a medial surface and a lateral surface, and having a foot having an arch and a heel and a toes portion, comprising:
a) at least one unitary stiff support structured and arranged to stiffly support the ankle in relation to the foot, wherein said at least one unitary stiff support comprises stiff portions comprising
i) at least one medial stiff portion structured and arranged to support the medial surface of the lower calf,
ii) at least one lateral stiff portion structured and arranged to support the lateral surface of the lower calf, and
iii) at least one arch stiff portion structured and arranged to support at least one substantial portion of the arch;
b) wherein said at least one unitary stiff support further comprises essentially no stiff portions structured and arranged to support the front surface of the lower calf, the rear surface of the lower calf, the top surface of the foot, nor the toes portion of the foot.

17. The system according to claim 16, wherein said at least one unitary stiff support further comprises essentially no stiff portions structured and arranged to support the heel.

18. The system according to claim 16, wherein said at least one unitary stiff support consists essentially of at least one plastic material.

19. The system according to claim 18, wherein said at least one unitary stiff support consists essentially of at least one thermoplastic material.

20. The system according to claim 19, wherein said at least one unitary stiff support consists essentially of at least one low-temperature-thermoplastic material.

21. The system according to claim 20, wherein said at least one unitary stiff support consists essentially of at least one low-temperature-thermoplastic material comprising substantial malleability at about 180 degrees Fahrenheit.

22. The system according to claim 18, wherein said at least one unitary stiff support comprises isoprene polymers.

23. The system according to claim 18, wherein said at least one unitary stiff support is approximately one eighth of an inch thick, before pre-forming.

24. The system according to claim 16, further comprising at least one inner lining structured and arranged to lie at least between essentially all of said unitary stiff support and the leg.

25. The system according to claim 24, wherein said at least one inner lining comprises at least one flexible material.

26. The system according to claim 25, wherein said at least one flexible material comprises at least one of the group consisting essentially of
a) fabric,
b) padded nylon fabric, and
c) leather.

27. The system according to claim 16, further comprising at least one outer lining, structured and arranged to lie at least outside of and covering essentially all of said at least one unitary stiff support.

28. The system according to claim 27, wherein said at least one outer lining comprises at least one flexible material.

29. The system according to claim 28, wherein said at least one flexible material comprises at least one of the group consisting essentially of
a) fabric,
b) padded nylon fabric, and
c) leather.

30. The system according to claim 16, further comprising:
a) at least one inner lining structured and arranged to lie at least between essentially all of said at least one unitary stiff support and the leg;
b) at least one outer lining, structured and arranged to lie at least outside of and covering essentially all of said at least one unitary stiff support;
c) at least one attacher structured and arranged to attach said at least one inner lining with said at least one outer lining in such manner as to hold essentially all of said at least one unitary stiff support within said at least one inner lining and said at least one outer lining.

31. The system according to claim 30, wherein said at least one attacher comprises at least one of the group consisting essentially of
a) stitches and
b) glue.

32. The system according to claim 16, further comprising at least one closure structured and arranged to adjustably tighten said semi-custom ankle brace system around the ankle.

33. The system according to claim 32, wherein said at least one closure comprises at least one of the group consisting essentially of
a) grommet and lace,
b) hook and loop fastener,
c) elastic closure, and
d) cinched strap.

34. The system according to claim 16, further comprising at least one tongue, structured and arranged to cover at least one gap under at least one closure of said at least one semi-custom ankle brace system.

35. The system according to claim 34, wherein said at least one tongue comprises at least one flexible material.

36. The system according to claim 35, wherein said at least one flexible material comprises at least one of at least one of the group consisting essentially of
a) fabric,
b) padded nylon fabric, and
c) leather.

37. A system, for stabilizing an ankle of a leg, having a lower calf and a foot having an arch and a heel and a toes portion, comprising:
a) at least one adjustably tightenable portion structured and arranged to essentially surround the lower calf; and
b) at least one other adjustably tightenable portion structured and arranged to essentially tighten around the ankle, the heel, and the arch;
c) wherein said at least one semi-custom ankle brace system is structured and arranged to be worn inside of at least one standard shoe large enough to hold said at least one semi-custom ankle brace system;
d) wherein said at least one semi-custom ankle brace system comprises at least one unitary stiff support structured and arranged to stabilize the ankle in relation to the foot in said at least one semi-custom ankle brace system; and
e) wherein said at least one unitary stiff support consists essentially of at least one low-temperature-thermoplastic material.

38. The system according to claim 37, wherein said at least one unitary stiff support consists essentially of at least one low-temperature-thermoplastic material comprising substantial malleability at about 180 degrees Fahrenheit.

39. The system according to claim 38, wherein said unitary stiff support comprises stiff portions comprising
a) at least one medial stiff portion structured and arranged to be placed essentially adjacent a medial surface of the lower calf,
b) at least one lateral stiff portion structured and arranged to be placed essentially adjacent a lateral surface of the lower calf, and
c) at least one arch stiff portion structured and arranged to be placed essentially adjacent at least one substantial portion of the arch;
d) wherein said at least one unitary stiff support further comprises essentially no stiff portions structured and arranged to be placed essentially adjacent a front surface of the lower calf, a rear surface of the lower calf, a top surface of the foot, nor the toes portion of the foot.

40. The system according to claim 39, wherein said at least one unitary stiff support further comprises essentially no heel stiff portion structured and arranged to be placed essentially adjacent to the heel.
